(19) United States
(12) Patent Application Publication
(10) Pub. No.: US 2021/0346578 A1
(43) Pub. Date: Nov. 11, 2021

(54) DEVICE AND METHOD FOR TREATING INFECTIONS

(71) Applicant: Watershed Medical, Inc., Bellevue, WA (US)

(72) Inventors: Clay NOLAN, Morgan Hill, CA (US); Christopher Ralph KINSELLA, Jr., Los Altos, CA (US)

(73) Assignee: Watershed Medical, Inc., Bellevue, WA (US)

(21) Appl. No.: 17/207,337

(22) Filed: Mar. 19, 2021

Related U.S. Application Data
(63) Continuation of application No. PCT/US2019/053549, filed on Sep. 27, 2019.
(60) Provisional application No. 62/737,754, filed on Sep. 27, 2018.
(60) Provisional application No. 62/905,881, filed on Sep. 25, 2019.

(51) Int. Cl. A61L 31/14; A61L 29/08; A61L 29/14

(52) U.S. Cl. CPC A61L 31/148; A61L 29/085; A61L 2400/12; A61L 2300/404; A61L 29/148

(57) ABSTRACT

Devices that are placed within fluid filled organs, such as the bladder, which reduce the risk of bacterial infections using a combination of active agents and mechanical disruption of bacteria.

### CROSS-REFERENCE TO RELATED APPLICATIONS

This application is continuation of PCT Application No. PCT/US2019/053549 filed Sep. 27, 2019, which claims the benefit of U.S. Application No. 62/737,754 filed Sep. 27, 2018, and also claims the benefit of U.S. Application No. 62/905,881 filed on Sep. 25, 2019, the entirety of each of which are incorporated by reference.

### FIELD OF THE INVENTION

Devices that are placed within fluid filled organs, such as the bladder, which reduce the risk of bacterial infections using a combination of active agents and mechanical disruption of bacteria.

### BACKGROUND OF THE INVENTION

Urinary tract infections (“UTI”) are infections in any part of the urinary system, such as the kidneys, ureter, bladder, and/or urethra. The UTI typically develops in the human bladder caused by the migration of bacteria into the bladder cavity through the urethra. UTI's are one of the most common types of infections that require medical treatment. Nearly, one in five adults develops a UTI and many of these individuals are at risk for a recurring infection.

The risks associated with UTI's can be significant for persons suffering from incontinence or for older persons, as well as with traumatic changes in the central nervous system, which are accompanied by urination disorders. In many cases, chronic urinary tract infections can require continuous medication, which, leads to progressive levels of resistance to antibiotics and ultimately to kidney problems.

UTI's and complications caused by UTI's are cost-intensive due to continuous need for medication and extended hospitalization and they can be life-threatening. In many cases, UTI's significantly reduce the quality of life, in particular when kidney damage leads to kidney failure requiring dialysis or implantation of a donor kidney.

In many cases, the UTI is only treated after the infection has developed and becomes symptomatic. However, current treatment to prevent the formation of UTI's requires continuous prophylactic oral intake of medication that can potentially strain the entire body, in particular with long-term medication. However, the increase of multi-drug resistant bacteria makes it harder to treat UTI's with a standard anti-biotic regimen as the UTI can recur since all of the bacteria causing the UTI are not eliminated.

The problems arising from bladder infections is that the infection can ascending to the kidney via the ureter. In many cases women are at greater risk of developing a UTI than are men. While the infection can be limited to the bladder and produce pain, serious consequences can occur if a UTI spreads to the kidneys or other organs as sepsis (urosepsis) can occur. The problem is increased in the elderly because UTI's are especially prevalent with incomplete voiding of the bladder, weakened immune systems, dehydration, and adult diapers. Where such factors increase the risk of UTI. In many cases elderly patients do not present with typical signs of infection until they become septic, at which point the patient requires hospitalization.

Therefore, there is a need to prevent and/or reduce the survival of bacteria within the bladder, where such an approach does not cause collateral risk to the patient. Such a treatment can provide benefits for patients at risk of a UTI. Such benefits are even more evident for incapacitated patients or the elderly, where the treatment eliminates the need for long term prophylactic administration of antibiotics and still reduces or eliminates a patient's risk of developing a UTI.

### BRIEF SUMMARY OF THE INVENTION

The illustrations and variations described herein are meant to provide examples of the methods and devices of the invention. It is contemplated that combinations of aspects of specific embodiments or combinations of the specific embodiments themselves are within the scope of this disclosure.

The present disclosure includes devices for reducing infections within a fluid filled organ using a combination of anti-bacterial properties and mechanical disruption of biofilms that would otherwise allow bacterial growth. The devices described herein also are configured to prevent encrustation of the device through deformation within the organ. In one example, the device comprises a carrier material formed into a fluid-permeable body such that the fluid can pass through the fluid-permeable body, the carrier material having a density that causes movement of the fluid-permeable body in the fluid of the fluid filled organ that mechanically disrupts formation of a biofilm to prevent bacteria from adhering to the biofilm; an active substance releasable from the carrier material, where the active substance comprises at least an anti-bacterial property such that when disposed in the fluid filled organ, the active agent enters the fluid to reduce bacteria or prevents bacterial growth within the fluid filled organ.

In one variation, the fluid-permeable body is compressible to permit delivery through a narrow conduit leading to the fluid filled organ.

Variations of the device can include a carrier material is selected from a group comprising a non-biodegradeable material, a biodegradeable material, and a combination thereof.

In additional variations, the fluid-permeable body is configured to produce turbulence in the fluid when moving in the fluid filled organ, where turbulence assist in mechanically disrupting the biofilm. In another variation, the carrier material comprises an open cell foam.

The device can be configured so that the fluid-permeable body is deformable such that movement of the fluid permeable body within the fluid filled organ causes deformation of the fluid-permeable body.

The device can further include an agent coated on the carrier material that prevents encrustation on the fluid-permeable body when positioned in the fluid filled organ. The agent can comprise a material selected from the group consisting of a urease inhibitor, a chelating agent, an antibacterial agent, an enzyme, and an inert coating.

In one variation, the carrier material comprises a varying density through a thickness of the carrier material, where the varying density increases deformation of the carrier material during contraction and function of the fluid-filled organ.

Variations of the device include the fluid-permeable body comprising a helical shape that conforms to a wall of the fluid-filled organ and deforms upon contraction of the fluid-filled organ.

The carrier material may comprise a plurality of discrete portions of carrier material constrained in a fluid permeable cover, wherein the fluid permeable cover prevents escape of the plurality of discrete portions of carrier material. The fluid-permeable cover can be degradable. In another variation, the device includes a neck filter configured for positioning at a neck of the fluid-filled organ to prevent escape of the carrier material from the fluid-filled organ.

In variations of the device, the fluid-permeable body is configured to fracture through an application of mechanical force. For example, the mechanical force comprises increased flow within the fluid-filled organ or a passage coupled to the fluid-filled organ. In another variation, the fluid-permeable body is configured to fracture through an application of a degradation agent. For example, the degradation agent can comprise a hydrolytic agent, an enzymatic agent, a chemical agent, and acidic urine.

The devices described herein can comprise a fluid-permeable body that is hydrophobic.

Another variation of the device comprises an assembly comprising a first ring, a second ring, and a plurality of rib structures; where the first ring has a first diameter and the second ring has second diameter; the plurality of rib structures each having a convex profile and each having an end coupled to the first ring and the second ring, such that the shape of the assembly is spherical, where each rib structure is spaced from an adjacent rib structure to allow the fluid to pass through the assembly; where the assembly is deformable such that contraction of the fluid-filled organ is sufficient to deform the assembly; where at least one of the plurality of rib structures produces an anti-bacterial effect when placed in the fluid.

In one variation, the first ring and the second ring comprise magnetic properties.

In an additional variation, each rib comprises a varying density along a length of the ring, such that the density increases towards a middle section of the rib.

Additional variations of the devices can include an outer body comprising a first cavity bound by a fluid impermeable surface and a first check valve permitting fluid flow into and out of the first cavity; and an inner body located within the first cavity, the inner body comprising a second cavity containing an agent configured to reduce bacteria within the bladder, the second cavity comprising a second check valve to permit flow of the agent into the first cavity; wherein expansion of the bladder causes the agent to flow through the second check valve into the first cavity, and where contraction of the bladder causes compression of the outer body to cause the agent to flow from the first cavity through the check valve and into the bladder.

The devices described herein are suitable for treatment of existing infections as well as for long term prophylactic administration to prevent occurrences of infection. However, unlike conventional prophylactic treatments, the current device does not present the drawbacks of current prophylactic anti-biotic treatments. Moreover, the implants disclosed herein can be intended for temporary, long term, or even permanent placement within the body.

### DESCRIPTION OF THE DRAWINGS

Each of the following figures diagrammatically illustrates aspects of the invention. Variation of the invention from the aspects shown in the figures is contemplated.

FIG. 1 illustrates a cross sectional view of a bladder showing the ureters terminating in utereric orifices within a cavity of the bladder as a catheter is advanced into the urethra to deploy an implant.

FIG. 2A shows a state where the implant is deposited within the cavity of the bladder.

FIG. 2B illustrates the implant floating within the bladder and causing turbulence within the urine.

FIG. 2C illustrates the implant adhering to a wall of the bladder as well as deforming under contraction of the bladder.

FIGS. 3A to 3C show variations of implants.

FIG. 4A illustrates another variation of an implant, where the implant comprises a ball shaped fluid permeable body.

FIG. 4B shows the implant of 4A in a compressed state.

FIGS. 6A and 6B show an implant that is assembled within the bladder.

FIGS. 7A and 7B illustrate a variation of an implant that delivers a therapy with each voiding or contraction of the muscles of the bladder.

### DETAILED DESCRIPTION

It is understood that the examples below discuss uses of an implant device for either treating a bladder for an existing infection in the urinary tract or for prophylactic administration to prevent an infection from occurring. However, unless specifically noted, variations of the device and method are not limited to use only in the bladder, instead, the device can be used for general surgical procedures to prevent infections from developing in fluid filled organs. For purposes of this disclosure, the term fluid-filled organ can be substituted for bladder. Therefore, the methods and device will have applicability in various parts of the body under any minimally invasive or invasive procedure. Moreover, the invention may be used in any procedure where the benefits of the method and/or device are desired. Furthermore, because it is impractical to display each and every combination of features and aspects of various embodiments, it is understood that where possible, every aspect or feature of an embodiment of the methods and/or devices can be combined with alternate embodiments of methods and/or devices.

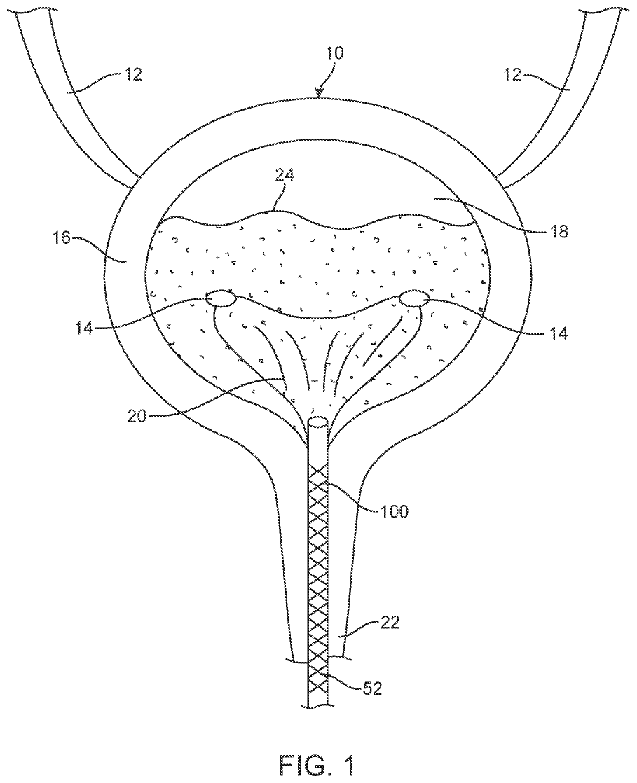

FIG. 1 illustrates a cross sectional view of a bladder 10 showing the ureters 12 terminating in utereric orifices 14 within a cavity 18 of the bladder 10. The bladder is surrounded by a muscle in a wall 16 of the bladder. In the illustrated variation, the bladder 10 contains urine 24 for purposes of illustration. As shown a catheter 52 or another introducer device is advanced into the urethra 22, where the catheter 52 contains an implant 100 as described herein. In the illustrated variation, the catheter 52 is advanced into the trigone 20 of the bladder 10 so that the device 100 can be deposited into the cavity 18 of the bladder 10. As discussed below, variations can include depositing the device 100 close to a wall 16 of the bladder 10 so that it adheres to the bladder wall 16. Alternatively, positioning the device 100 into the bladder 10 can occur without a catheter, surgically, or through an endoscopic approach.

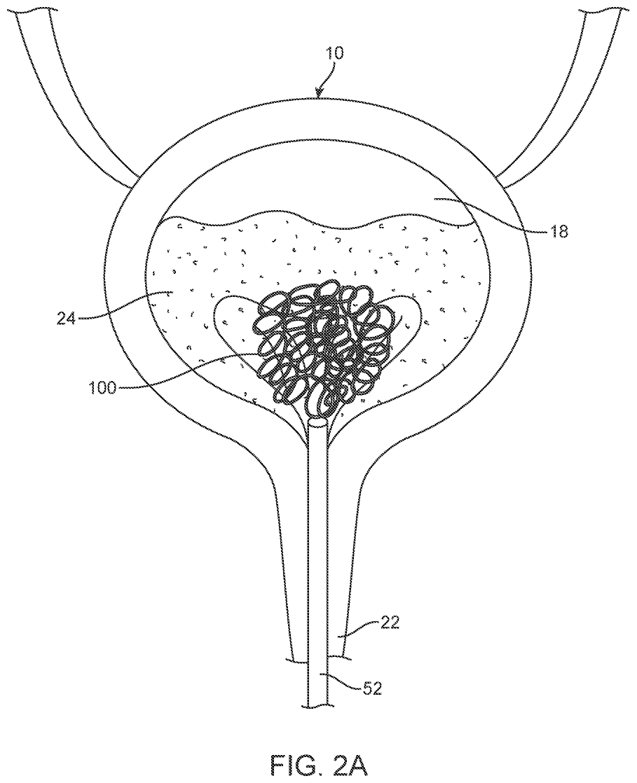

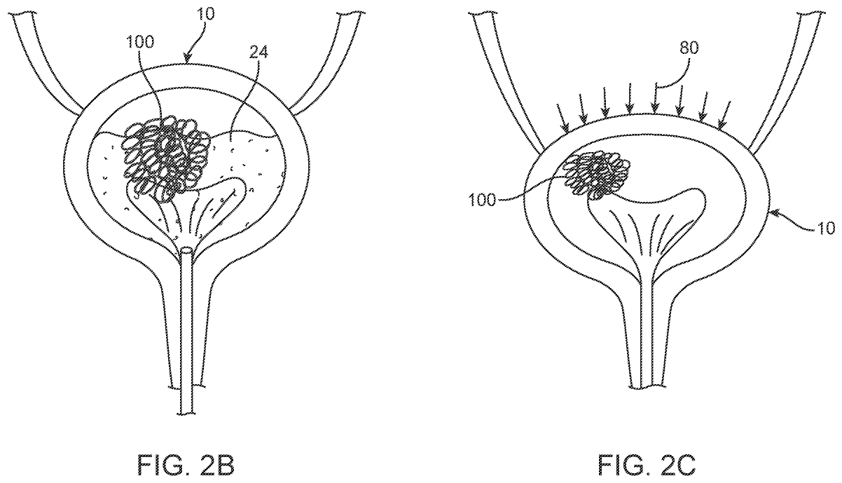

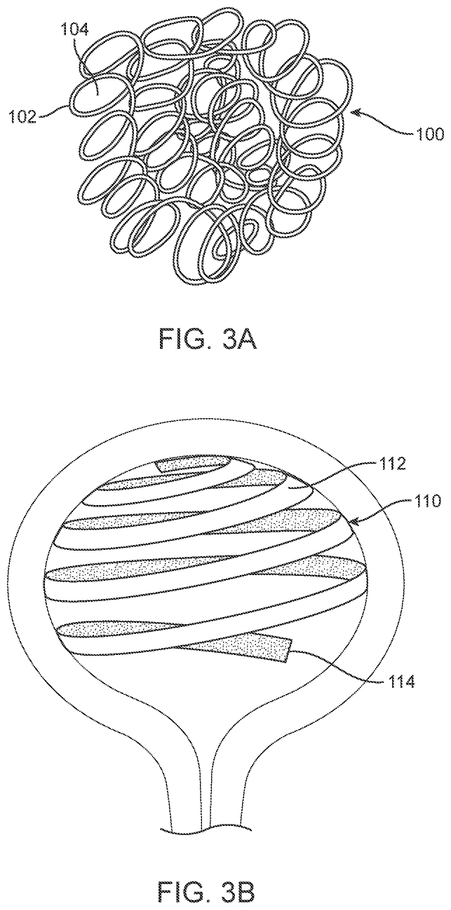

FIG. 2A shows a state where the temporary implant 100 is deposited within the cavity 18 of the bladder 10. As shown and discussed in additional detail below, the temporary implant 100 accomplishes several objectives. For example, the device 100 is permeable, meaning that urine 24 (or other fluid) is not blocked from exiting the bladder 10 via the urethra. The device 100 assists in preventing infection within the bladder 10 by preventing or disrupting the growth of bacteria within the bladder 10. In one variation, the device 100 performs a bacterial agitation within the bladder 10 or along a lining of the bladder. This bacterial agitation disrupts adherent bacteria and disrupts biofilms using a mechanical action.

A biofilm typically comprises any syntrophic consortium of microorganisms in which cells stick to each other or to a surface. These cells become embedded within an extracellular matrix that is composed of extracellular polymeric substances. Because the matrix has a three-dimensional structure it provides a means for microbes and other bacteria to grow and multiply. By disrupting formation of biofilms in the bladder, the present devices 100 can reduce or eliminate bacteria growth within the bladder. Alternatively, the implants can maintain the bacteria at levels that are unable to cause a UTI.

Once the biofilms are mechanically disrupted, an active agent can be released into the bladder 10 to treat bacteria or to prevent re-adherence. The implant 100 shown in FIG. 2 illustrates a carrier material that can comprise an open cell foam (such as V.A.C. GranuFoam™ supplied by KCI-medical), where the open cell foam comprises one or more strands wound together to form a fluid permeable body. The fluid permeable body can mechanically disrupt biofilms forming in the bladder through mechanical action. Furthermore, the foam carrier material can include an active agent such as described herein and serve as a delivery mechanism for the active agent. In one variation, the active agent is a silver or anti-biotic that further resists formation of bacteria. Alternatively, the carrier material can comprise any matrix structured material that can contain or be coated with an active substance while also providing the properties required of the implants disclosed herein. In additional variations of the device the active substance or agent is inherent to the carrier material as opposed to the agent being coated, embedded, deposited, etc. on the body. For example, in those variations where the carrier material is a silver-based material, the active substance/agent comprises silver ions that are released as the silver oxidizes. Un variations where the carrier material comprises a polymer with antibiotic properties, the carrier material comprises the polymer and the active substance is part of the carrier material. In each case, the active substance is released from the carrier material to eliminate bacteria or at least reduce it to a level that does not pose an infection risk.

Another benefit to using a fluid permeable body for the implant 100 is that ordinary movement of the implant 100 within the bladder 100 can cause turbulence in the urine, which is another means to prevent formation of biofilms that foster the growth of bacteria.

In one variation, an implant device can be made from a non-biodegradable material (e.g. polyurethane) with coatings or other active agent materials as described herein. In another variation, the device body is made from a biodegradable material (PGLA, PLLA, Polycaprolactone, or mixtures thereof) with various active agents as described herein. In another variation, the device is fabricated from a combination of biodegradable and non-biodegradable material with coatings or active agents as described below.

In one variation of the implant, the carrier material forming the implant 100 can have a density that causes movement of the fluid-permeable body in the urine, where such movement mechanically disrupts formation of a biofilm to prevent bacteria from adhering to the carrier material. For example, as shown in FIG. 2B, the implant 100 can be configured to be less dense than the urine 24 such that the implant 100 float within the bladder 24 and causes turbulence within the urine 24, which works against formation of any biofilm. FIG. 2C illustrates the implant 100 adhering to a wall of the bladder 10 as well as deforming under contraction (as illustrated by contractile action 80) of the bladder 10. The implants can have mucoadhesive properties that allow it to stick to the wall of the organ. Alternatively, the implants can have structural features that cause the implant to remain attached to a wall of the organ.

Variations of the implants described herein will be deformable to allow deployment of the implant as well as deformation of the implant such that movement of the implant within the bladder or as a result of compression of the bladder, will deform the fluid permeable body (and/or the carrier material) to prevent formation of micro-organisms that would otherwise cause encrustation of the implant while in the bladder. Encrustation typically occurs in when urine is alkaline such that crystals form on surfaces within the urine. Such encrustation is commonly associated with indwelling catheters positioned within a urethra, where the encrustation obstructs the flow of urine.

The implant 100 shown in FIGS. 2A and 2B is only one example of implants under the present disclosure. As discussed below, the implants under the present disclosure can comprise any number of structural features that allow for a porous body to permit flow therethrough, deformation under the environment of the bladder, movement within the bladder, and optionally adherence to a wall of the bladder. Such features are intended to mechanically disrupt formation of biofilms that are conducive to bacterial growth, reduction of encrustation of the implant, and release of an active agent to ensure prevention of a UTI from forming.

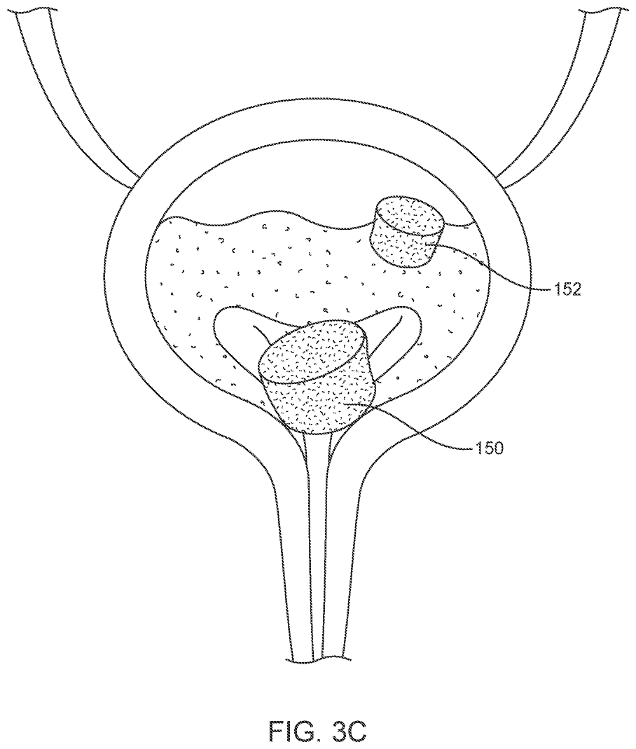

FIG. 3A illustrates the implant 100 of FIGS. 2A and 2B. As shown, the implant comprises a carrier material 102 that forms a fluid permeable body that permits flow therethrough. The carrier material 102 can comprise an open cell foam or any material disclosed herein. In certain variations, the carrier material and/or construction of the implant 100 permits flotations of the implant 100 within the urine of the bladder. As noted above, the mechanical interaction of the implant 100 with the urine and bladder walls assist in reducing formation of bacteria. The implant 100 is compressible (e.g., the permeable body is compressible and/or the carrier material is flexible), where such compressibility permits delivery of the implant 100 into the bladder. The compressible nature of the implant 100 also permits deformation of the implant when within the bladder to further assist in mechanically disrupting biofilms that would otherwise contribute to encrustation of the implant.

FIG. 3B illustrates another variation of an implant 110 comprising a carrier material 112 that is shaped to encourage apposition to a bladder wall. For example, the implant 110 can comprise a pre-set shape such that upon deployment into the bladder, the implant 110 engages the wall of the bladder. Alternatively, the implant 110 can be configured to adhere to the bladder wall as it is deployed within the bladder. In any case, the implant 110 will be constructed such that contraction of the bladder causes deformation of the implant 110 during voiding. Again, the implant body 110 is constructed to be fluid permeable such that fluid can pass therethrough. Moreover, a portion of the implant 110 (e.g., the end portion 114) can move through fluid/urine within the bladder to encourage turbulence of the urine within the bladder.

FIG. 3C is intended to illustrate yet another variation of an implant 150, where the implant body is constructed from an open cell foam that has a porosity, which allows fluid to pass through the body of the implant 150. FIG. 3C also illustrates that the implant 150 (as well as any other implant disclosed herein) comprises a density that does not float within the bladder. Moreover, FIG. 3C illustrates that more than one implant 150 and 152 can be positioned within the bladder where the implants 150 and 152 can have different characteristics.

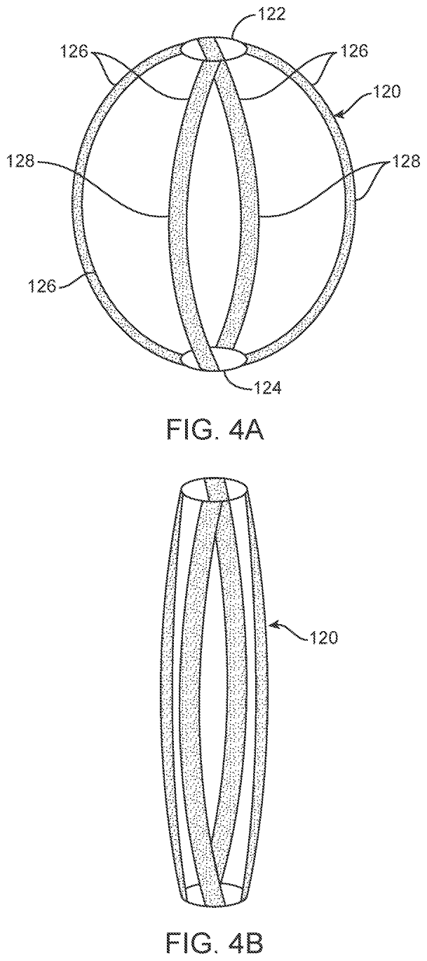

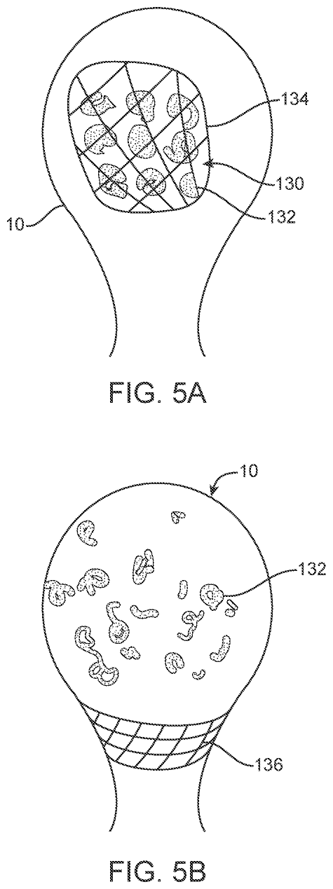

FIG. 4A illustrates another variation of an implant 120, where the implant comprises a ball shaped fluid permeable body formed form a first ring 122, a second ring 124, and a plurality of arms 126 coupled to each ring 122, 124. The implant 120 can be constructed from any material. For example, the implant 120 comprises a metal alloy where the arms 126 comprise a metal (such as silver or copper) to provide antibiotic effects. The rings 122 and 124 can be used to align the device 120 for insertion to and removal from the bladder. For instance, the rings can comprise magnetic properties for alignment with a catheter or urethra. FIG. 4B illustrates the implant 120 in a compressed or deformed configuration. Such a configuration allows for positioning in a catheter on a mandrel for deployment/removal from the body. FIG. 4A also illustrates a feature of the implant where the density of the arm 126 along a mid-portion 128 is greatest. This configuration promotes bending of the implant across an entire length when pressure is applied. This bending prevents encrustation on the device. In one example, the density is varied along a length of the arms so the portion of the arm most exposed to force (e.g., the mid portion 128) have a higher density than the materials at the ends (e.g., adjacent to the rings).

FIG. 5A illustrates another variation of an implant 130, which comprises multiple primary devices 132 that are formed from the carrier materials disclosed herein. In this variation, the primary carrier material devices 132 are positioned into the bladder 10 and retained within a second device 134. Once the second device is removed or degrades, the primary devices 132 pass through the urethra. The removal mechanism can occur by natural voiding of the bladder or mechanically using a tool (e.g., a snare, suction, catheter, etc.) As shown, the primary devices can be held in close proximity by the second device 134 so that compression by the bladder 10 results in deformation of the entire implant 130 as well as each primary device 132.

FIG. 5B illustrates another variation of an implant comprising two separate components. In this variation, a secondary device 136 prevents migration of the primary carrier material bodies 132 from the bladder 10. The secondary device 136 can be removed mechanically or through degradation (e.g., chemical, enzymatic, or over time).

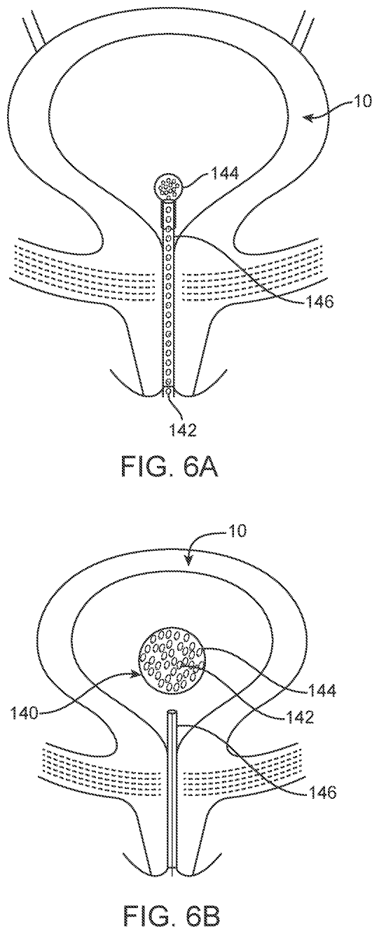

FIG. 6A shows another variation of an implant that is delivered into the bladder 10 using a catheter 146 with a retaining member 144 that can expand in profile. The member 144 can be distensible, non-distensible, or simply a basket/netting/sheath that can be compressed or wrapped for delivery but increases in profile as the carrier material enters. The catheter 146 delivers a carrier material 142 into the expandable member causing expansion of the retaining member 144 to form the implant 140. The catheter 146 can be disengaged from the implant 140 and removed from the urethra and bladder. The expanded implant 140 will not migrate through the urethra and is still fluid permeable. Variations of the implant 140 float or have other features disclosed herein. After a designated amount of time, the retaining member 144 degrades or is fractured, which releases the carrier material 142. The size of the carrier material allows fluid migration from the bladder through the urethra.

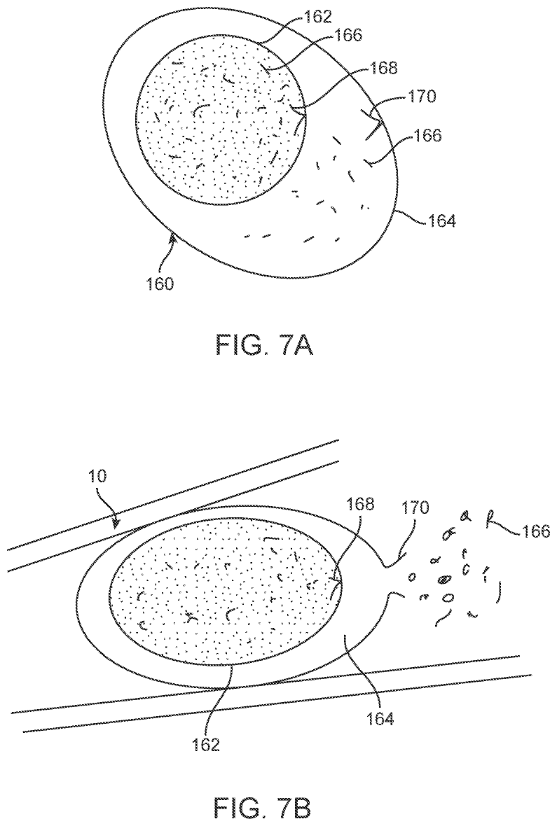

FIGS. 7A and 7B illustrate a variation of an implant 160 that delivers a therapy with each voiding or contraction of the muscles of the bladder. In the illustrated example, as show in FIG. 6A, an inner chamber 162 of the device is somewhat rigid and filled with a concentrated therapy or carrier material 166. The inner chamber 162 is encapsulated by a larger outer, flexible material or housing 164. The two interior spaces of each chamber 162 and 164 are connected by a check valve 168 that, in certain conditions, allows the contents of the inner chamber 162 to flow into the outer chamber 164. The outer chamber 164 includes an exterior check valve 170 that fluidly couples the contents of the outer chamber 164 to the reservoir of a bladder. When the bladder 10 contracts, as shown in FIG. 7B, the walls of the bladder compress the outer chamber 164 forcing any material 166 therein to exit into the bladder. When the bladder relaxes, the outer chamber 164 expands and the pressure differential between the inner chamber 162 and outer chamber 164 opens the inner check valve 168 causing migration of the contents 166 of the inner chamber 162 to escape to the outer chamber 164, which reloads the next dose into the outer chamber 164.

In those variations of implants that require degradation, fracturing of the implant can occur through mechanical compression (i.e. cycling of bladder emptying), fracturing of device through other mechanical action (e.g., snare, suction, scissors, etc), hydrolytic degradation in aqueous environment, introduction of enzymatic or chemical agent into bladder, environment (e.g. bladder irrigation with hydrogen peroxide or naturally over time), systemic administration of agent which changes composition of urine to trigger degradation (e.g. methenamine hippurate to acidify urine). Moreover, the carrier materials used herein can be configured such that if they become stuck in the urethra or neck of the bladder, the increased flow or turbulence of the urine attempting to flow through a restricted area will cause increased or turbulent flow. In such cases, the carrier material can be selected such that it breaks down or erodes when subject to increased flow or turbulence.

The variations of the implants described herein can contain any number of active agents that ultimately reduce bacterial formation or re-adherence within the organ. The active agents can comprise coatings and/or can be embedded within a matrix of the carrier material that forms the implant whereby the matrix releases the agent (e.g. granules). Some examples of such agents includes the following (as well as combinations of various agents). In one variation, the active agent can comprise one or more substances that control pH of the urine to prevent crystallization of solute onto the device body; substances that prevent bacterial enzyme urease from activity; urease inhibitor e.g. N-(n-butyl) thiophosphoric triamide (NBPT). The agent can also decrease effective solutes for crystallization within the urine. For example, the matrix or device body can be coated with chelating agents, e.g. ethylenediaminetetraacetic acid (EDTA), dimercaptosuccinic acid, dimercaprol, penicillamine. The agent can decrease bacterial activity to prevent encrustation of the carrier material or device body. For example, the carrier material can be coated with antibacterial agents, a compound containing silver e.g. (silver nitrate, elemental silver, etc.), another substance such as an antibiotic drug, copper, zinc, gold, antibacterial nanoparticles, liposomes, aptamers, dendrimers, antimicrobial peptides, inorganic or polymeric nano-particles, smart nanoparticles. etc. The active agent can comprise a coating that prevents the formation of a biofilm. A biofilm typically comprises any syntrophic consortium of microorganisms in which cells stick to each other or to a surface. These cells become embedded within an extracellular matrix that is composed of extracellular polymeric substances. Because the matrix has a three-dimensional structure it provides a means for microbes and other bacteria to grow and multiply. By disrupting formation of biofilms in the bladder, the present devices 100 can reduce or eliminate bacteria growth within the bladder. Alternatively, the implants can maintain the bacteria at levels that are unable to cause a UTI.

For example, such coatings include enzymes that degrade the biofilm extracellular matrix, such as dispersin B and deoxyribonuclease, nitric oxide, etc. The agent can include materials and/or coatings that decrease the affinity of crystals from attaching to the device (e.g., polyurethane, PTFE)

As for other details of the present invention, materials and manufacturing techniques may be employed as within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described. For example, the invention includes combinations of aspects of the variations of the devices described herein as well as the combination of the variations themselves. Also, any optional feature of the inventive variations may be set forth and claimed independently, or in combination with any one or more of the features described herein. Accordingly, the invention contemplates combinations of various aspects of the embodiments or combinations of the embodiments themselves, where possible. Reference to a singular item, includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments, or the embodiments themselves can be combined. Where such combinations are intended to be within the scope of this disclosure.

We claim:

1. A device for reducing infections within a fluid filled organ, the device comprising:
a carrier material formed into a fluid-permeable body such that the fluid can pass through the fluid-permeable body, the carrier material having a density that causes movement of the fluid-permeable body in the fluid of the fluid filled organ that mechanically disrupts formation of a biofilm to prevent bacteria from adhering to the biofilm; and
an active substance releasable from the carrier material, where the active substance comprises at least an anti-bacterial property such that when disposed in the fluid filled organ, the active agent enters the fluid to reduce bacteria or prevents bacterial growth within the fluid filled organ.

2. The device of claim 1, where the fluid-permeable body is compressible to permit delivery through a narrow conduit leading to the fluid filled organ.

3. The device of claim 1, wherein the carrier material is selected from a group comprising a non-biodegradeable material, a biodegradeable material, and a combination thereof.

4. The device of claim 1, wherein the fluid-permeable body is configured to produce turbulence in the fluid when moving in the fluid filled organ, where turbulence assist in mechanically disrupting the biofilm.

5. The device of claim 1, wherein the carrier material comprises an open cell foam.

6. The device of claim 1, wherein the fluid-permeable body is deformable such that movement of the fluid permeable body within the fluid filled organ causes deformation of the fluid-permeable body.

7. The device of claim 1, further comprising an agent coated on the carrier material that prevents encrustation on the fluid-permeable body when positioned in the fluid filled organ.

8. The device of claim 7, wherein the agent comprises a material selected from the group consisting of a urease inhibitor, a chelating agent, an antibacterial agent, an enzyme, and an inert coating,

9. The device of claim 1, wherein the carrier material comprises a varying density through a thickness of the carrier material, where the varying density increases deformation of the carrier material during contraction and function of the fluid-filled organ.

10. The device of claim 1, wherein the fluid-permeable body comprises a helical shape that conforms to a wall of the fluid-filled organ and deforms upon contraction of the fluid-filled organ.

11. The device of claim 1, wherein the carrier material comprises a plurality of discrete portions of carrier material constrained in a fluid permeable cover, wherein the fluid permeable cover prevents escape of the plurality of discrete portions of carrier material.

12. The device of claim 11, wherein the fluid-permeable cover is degradable.

13. The device of claim 1, further comprising a neck filter configured for positioning at a neck of the fluid-filled organ to prevent escape of the carrier material from the fluid-filled organ.

14. The device of claim 1, wherein the fluid-permeable body is configured to fracture through an application of mechanical force.

15. The device of claim 14, wherein the mechanical force comprises increased flow within the fluid-filled organ or a passage coupled to the fluid-filled organ.

16. The device of claim 1, wherein the fluid-permeable body is configured to fracture through an application of a degradation agent.

17. The device of claim 15, where the degradation agent comprises a hydrolytic agent, an enzymatic agent, a chemical agent, and acidic urine.

18. The device of claim 1, wherein the fluid comprises urine and the fluid-permeable body is configured to float when placed within the urine.

19. The device of claim 1, wherein the fluid-permeable body is hydrophobic.

20. A device for reducing infections within a fluid filled organ, the device comprising:
an assembly comprising a first ring, a second ring, and a plurality of rib structures;
where the first ring has a first diameter and the second ring has second diameter;
the plurality of rib structures each having a convex profile and each having an end coupled to the first ring and the second ring, such that the shape of the assembly is spherical, where each rib structure is spaced from an adjacent rib structure to allow the fluid to pass through the assembly;
where the assembly is deformable such that contraction of the fluid-filled organ is sufficient to deform the assembly; and
where at least one of the plurality of rib structures produces an anti-bacterial effect when placed in the fluid.

21. The device of claim 20, where the first ring and the second ring comprise magnetic properties.

22. The device of claim 20, where each rib comprises a varying density along a length of the ring, such that the density increases towards a middle section of the rib.

23. The device of claim 20, wherein the assembly is configured to fracture through an application of mechanical force.

24. The device of claim 23, wherein the mechanical force comprises increased flow within the fluid-filled organ or a passage coupled to the fluid-filled organ.

25. The device of claim 20, wherein the assembly is configured to fracture through an application of a degradation agent.

26. The device of claim 25, where the degradation agent comprises a hydrolytic agent, an enzymatic agent, a chemical agent, and acidic urine.

27. The device of claim 20, wherein the fluid comprises urine and the assembly is configured to float when placed within the urine.

28. The device of claim 27, wherein the assembly is hydrophobic.

29. A device for reducing infections within a fluid filled organ, the device comprising:
an outer body comprising a first cavity bound by a fluid impermeable surface and a first check valve permitting fluid flow into and out of the first cavity; and
an inner body located within the first cavity, the inner body comprising a second cavity containing an agent configured to reduce bacteria within the bladder, the second cavity comprising a second check valve to permit flow of the agent into the first cavity; and
wherein expansion of the bladder causes the agent to flow through the second check valve into the first cavity, and where contraction of the bladder causes compression of the outer body to cause the agent to flow from the first cavity through the check valve and into the bladder.
